(19) United States
(12) Patent Application Publication
(10) Pub. No.: US 2018/0000738 A1
(43) Pub. Date: Jan. 4, 2018

(54) NOVEL SPRAY NOZZLE AND PROCESS FOR MAKING NANOPARTICLES

(71) Applicant: Bend Research, Inc., Bend, OR (US)

(72) Inventors: Vikram J. Pansare, Bloomington, IN (US); Aaron Keith Goodwin, Bend, OR (US); Ronald Arthur Beyerinck, Sisters, OR (US); Robert K. Prud'homme, Princeton, NJ (US); Dwayne Thomas Friesen, Bend, OR (US); David Thomas Vodak, Bend, OR (US)

(73) Assignee: Bend Research, Inc., Bend, OR (US)

(21) Appl. No.: 15/520,812

(22) PCT Filed: Oct. 5, 2015
(86) PCT No.: PCT/IB2015/057600

§ 371 Date: Apr. 20, 2017

Related U.S. Application Data
(60) Provisional application No. 62/067,581, filed on Oct. 23, 2014.

(51) Int. Cl. A61K 9/16; B05B 1/00; B05B 7/24

(52) U.S. Cl. CPC A61K 9/1694; B05B 7/2489; A61K 9/1623; B05B 1/005

(57) ABSTRACT

Methods for making particulate material include providing a first solution comprising one or more solvents and an active agent, providing a second solution comprising an antisolvent, mixing the first solution with the second solution to form a mixture, atomizing the mixture with a gas to produce droplets, and drying the droplets by directing the droplets into a chamber for removal of the solvent and the antisolvent to produce solid particles. Various apparatuses for producing particulate material in this manner are also provided.

### FIELD

This disclosure relates to equipment and processes for forming compositions comprising small domains of active agent and a matrix, and methods of using them.

### BACKGROUND

A number of methods have been used to form particulate constructs for use in pharmaceutical products. Examples of such methods include the spray dry dispersion (SDD) method, such as that described in U.S. Pat. No. 8,431,159 B2. Such methods have been found effective for generating particulate material particularly from hydrophobic active agents (e.g. agents with high log P values), by atomization and rapid drying of hydrophobic active agents solubilized with semi-hydrophobic matrix phases such as hydroxypropylmethylcellulose (HPMC) or hydroxypropylmethylcellulose acetate succinate (HPMCAS). However, a need still remains for an apparatus and process that more effectively generates solid particles from active agents with low log P values and high ratios of Tm/Tg (melting temperature and glass transition temperature respectively).

US 2004/0091546 exemplifies an apparatus and process utilizing a combination of chemistry and kinetics to enable the formation of amorphous, or nearly amorphous, phase active agents, which display high active agent supersaturations upon dissolution. The formation of nanoparticles is induced by mixing at least one process solvent comprising amphiphilic copolymers with at least one nonprocess solvent capable of changing the charge of the local molecular environment of the amphiphilic copolymers. The process solvent(s) or non-process solvent(s) can further contain an additive target molecule useful for a specific indication which can be coprecipitated or coated with the amphiphilic copolymer. The process solvent or non-process solvent can also contain supplemental additives useful for the production or subsequent use of the nanoparticles. Following mixing, post processing can be applied to eliminate the solvent leaving the solid nanoparticles.

However, although such process is capable of producing controlled size, polymer-stabilized and protected nanoparticles of hydrophobic organics at high loadings and yields, it still typically results in active agent domains that can increase in size, or even crystallize into larger crystals following a phenomena known as Ostwald ripening.

Thus there still remains a need for an apparatus and process that effectively overcomes the problems of the prior art.

### SUMMARY

In a first aspect, the present disclosure relates to a method for making particulate material comprising the steps of: providing a first solution comprising one or more solvents and active agents; providing a second solution comprising an antisolvent, wherein the first solution and/or the second solution further comprises a matrix material; mixing said first solution with said second solution, to form a mixture typically in the form of a nano-precipitate; atomizing the mixture with a gas to produce droplets; drying said droplets by directing said droplets into a chamber for removal of at least a portion of said solvent and said antisolvent, to produce solid particles; wherein said atomizing step occurs substantially immediately after said mixing step and is followed by said drying step in a continuous mode, and wherein the mixing step starts at a dedicated position of a spray-drying nozzle.

In a second aspect, the present disclosure relates to a spray-drying nozzle, for use in the production of particulate material, comprising: a first conduit, having an inlet and an outlet, for conducting a first solution typically comprising a solvent, an active agent and a matrix material; a second conduit, having an inlet and an outlet, for conducting a second solution typically comprising an antisolvent; a mixing chamber being arranged to permit mixing of the first and second solution to generate a mixture typically in the form of a nano-precipitate, wherein said mixing chamber is in fluid communication with both said first and second conduits and is located proximal to the outlets thereof; one or more gas conduit(s), each having an inlet and an outlet, in fluid communication with an atomization region of the nozzle and preferably not being in direct fluid communication with the mixing chamber, wherein the atomization region is in fluid communication and downstream said mixing chamber and proximal to an outlet orifice of the nozzle, and wherein the gas conduit(s) are arranged to provide a gas flow into the atomization region to atomize said mixture into droplets that are jetted out of the spray-drying nozzle from said outlet orifice in the form of a spray, typically wherein the diameter of said outlet orifice is sized such that the mixture undergoes jetting when exiting said outlet orifice

### BRIEF DESCRIPTION OF THE DRAWINGS

FIG. 1 is a schematic drawing of a section of a spray nozzle according to one embodiment of the present disclosure.

### DETAILED DESCRIPTION

The present disclosure relates to equipment and processes for forming compositions comprising a plurality of distinct domains of active agent and a matrix, and methods of using them. Various embodiments are disclosed herein. Although methods and materials similar or equivalent to those described herein can be used in the practice of the present technology, only certain suitable methods and materials are described herein. The following description is exemplary in nature and is not intended to limit the scope, applicability, or configuration of the disclosure(s) in any way. Various changes to the described embodiments may be made, such as in the function and arrangement of the elements described herein, without departing from the scope of the disclosure(s).

As used in this application and in the claims, the singular forms “a,” “an,” and “the” include the plural forms unless the context clearly dictates otherwise. Additionally, the term “includes” means “comprises.” Further, the term “coupled” generally means electrically, electromagnetically, and/or physically (e.g., mechanically or chemically) coupled or linked and does not exclude the presence of intermediate elements between the coupled or associated items absent specific contrary language.

The term “nano-precipitate” or “nano-precipitation” as used herein means a precipitation of finely dispersed active agent in a mixture, in the form of sub-micron particles.

The term “jetted” or “jetting” as used herein means that the element referred to is projected into a surrounding medium (e.g. a gas) in the form of a stream, usually from some kind of nozzle, aperture or orifice. Typically the stream may travel a given distance from an orifice prior to dissipating into the surrounding medium.

The term “continuous mode” as used herein means that the steps referred to occur in direct succession in an in-line process arrangement, typically with negligible waiting times between process steps.

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods, as known by those of ordinary skill in the art. When directly and explicitly distinguishing embodiments from discussed prior art, the embodiment numbers are not approximates unless the word “about” is recited.

In one embodiment, a method for making particulate material comprises the steps of: providing a first solution comprising one or more solvents, and active agents; providing a second solution comprising an antisolvent; wherein the first solution and/or second solution, typically the first solution, further comprises a matrix material; mixing said first solution with said second solution, to form a mixture; atomizing the mixture with a gas to produce droplets; drying said droplets by directing said droplets into a chamber for removal of at least a portion of said solvent and said antisolvent, to produce solid particles; wherein said atomizing step occurs substantially immediately after said mixing step and is followed by said drying step in a continuous mode, and wherein the mixing step starts at a dedicated position of a spray-drying nozzle. An advantage of this method is that the residence time of the particles formed by nano-precipitation during mixing of the first and second solutions is greatly reduced. This means that essentially immediately after nano-precipitation the mixture is broken down into small droplets in the form of a spray and dried immediately thereafter. This ensures negating particle size increases, under the so called “Ostwald ripening” phenomenon, otherwise typically associated with higher residence times. Such further results in nicely distributed active-rich and active-poor domains in the resulting particles. A further advantage is compactness of the execution and the possibility of utilizing the same spray drying unit for both standard spray drying and the spray drying method of the present disclosure simply by replacing the nozzle of the unit, thus further reducing turnaround times. The first solution may flow into the mixing chamber at a first flow rate and the second solution may flow into the mixing chamber at a second flow rate, wherein the first and second flow rates are both greater than 0 mL/min and are different (i.e. not the same flow rate), preferably wherein the second flow rate is greater than the first flow rate, more preferably wherein the second flow rate is at least 2 times, preferably at least 3 times, more preferably at least 4 times, the first flow rate. An advantage of this arrangement is more effective mixing of the first and second solutions within the nozzle configuration and for generating the desired turbulence for rapid nano-precipitation prior to atomization of the mixture.

In a further embodiment, a spray-drying nozzle, for use in the method described above, comprises: a first conduit, having an inlet and an outlet, for conducting a first solution typically comprising a solvent, an active agent and optionally a matrix material; a second conduit, having an inlet and an outlet, for conducting a second solution typically comprising an antisolvent and optionally a matrix material; a mixing chamber, typically substantially funnel-shaped, being arranged to permit mixing of the first and second solution to generate a mixture generally by static mixing, wherein said mixing chamber is in fluid communication with both said first and second conduits and is located proximal to the outlets thereof; one or more gas conduit(s), each having an inlet and an outlet, in fluid communication with an atomization region of the nozzle, wherein the atomization region is in fluid communication and downstream said mixing chamber and proximal to an outlet orifice of the nozzle, and wherein the gas conduit(s) are arranged to provide a gas flow into the atomization region to atomize said mixture into droplets that are jetted out of the spray-drying nozzle from said outlet orifice in the form of a spray, typically wherein the diameter of said outlet orifice is sized such that the mixture undergoes jetting when exiting said outlet orifice. The nozzle may be in the form of a detachable unit typically such that it may conveniently be replaced with a standard spray-drying nozzle when needed. Advantages of a nozzle arrangement as described include: a simple and effective way of achieving the lowest possible residence times described above, interchangeability and modularity within the spray-drying unit, compactness of the design for achieving both mixing and atomization within the same replaceable component. Preferably, a first flow rate of the first solution flowing through the first conduit may be different from a second flow rate of the second solution flowing through the second conduit, preferably wherein the second flow rate is greater than the first flow rate, more preferably wherein the second flow rate is at least 2 times, preferably at least 3 times, more preferably at least 4 times, the first flow rate.

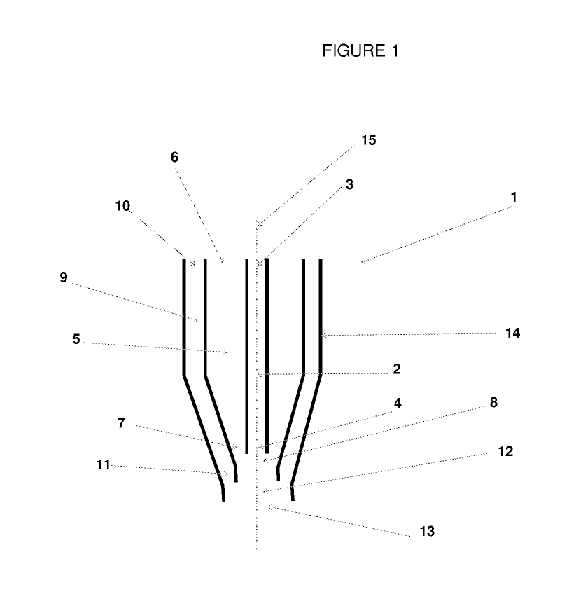

FIG. 1 shows a schematic representation of a suitable nozzle for the process. The nozzle can include an inner conduit that conducts a solvent solution with an active agent and optionally a matrix material, one or more adjacent conduits that conducts water or an antisolvent and optionally, a matrix material, and one or more outer conduits that conducts a gas for atomization. The antisolvent conduit(s) can be annularly disposed around the inner conduit, or can be arranged in non-annular geometries adjacent to the inner tube. Similarly, the outer gas conduit(s) can be positioned annularly around the antisolvent conduit(s) or otherwise. The inner solvent conduit joins with the adjacent antisolvent conduit(s) at an initial mixing region where the solvent solution mixes with the antisolvent. The mixing chamber can have a tapered shape moving from the inlet conduits to narrow outlet conduit. The overall cross-section of the conduits is reduced at this mixing region to create a flow of the mixture through the outlet conduit, which then is rapidly atomized by the gas flow to form droplets that exit the nozzle into the spray-drying chamber (not shown in FIG. 1). The residence time between the mixing of the solvent solution with the antisolvent and the atomization is typically very short, such as less than 500 milliseconds (ms), less than 250 ms, less than 150 ms, less than 110 ms, less than 100 ms, less than 90 ms, less than 80 ms, less than 70 ms, and/or less than 60 ms.

In an embodiment, the distance between the mixing chamber and the outlet orifice of the nozzle if from 0 to 10 cm, preferably from 0.5 to 8 cm, more preferably from 1 to 5 cm, even more preferably from 1 to 3 cm.

In the embodiments of FIG. 1, the mixing chamber where precipitation occurs is within the housing of the nozzle and positioned just before the atomization portion of the nozzle. “Within the housing of the nozzle” or “within the nozzle” means within a common rigid structure or housing that is identifiable as the nozzle or part of the nozzle, as opposed to the mixing chamber being located apart from the nozzle structure and coupled thereto by a conduit.

In exemplary embodiments, a nozzle can be formed by modifying a two-fluid atomizer by inserting poly ether ether ketone (PEEK) tube, such as a HPLC PEEK tube, into the central fluid delivery space. The inner tube can have an inside diameter ranging from 100 μm to 0.25 mm in some embodiments.

It is understood that the method herein may be carried out with a spray nozzle according to any of the embodiments described herein.

Active Agents

Embodiments of the disclosed process are suitable for use with any biologically active compound desired to be administered to a patient in need of the active agent. The compositions may contain one or more active agents. As used herein, by “active” or “active agent” is meant a drug, medicament, pharmaceutical, therapeutic agent, nutraceutical, or other compound that may be desired to be administered to the body. The active may be a “small molecule,” generally having a molecular weight of 3000 Daltons or less.

The active agent may be highly water soluble (i.e., greater than 30 mg/mL at 25° C.), sparingly water soluble (i. e., 5-30 mg/mL), or a low-solubility active agent (i.e., less than 5 mg/mL). In one embodiment, the active agent is a “low-solubility active agent,” and the active agent has a solubility in water (at 25° C.) of less than 5 mg/mL. In another embodiment, the active agent may have an even lower water solubility, such as less than 1 mg/mL, less than 0.1 mg/mL (100 μg/mL), less than 0.01 mg/mL (10 μg/mL), and even less than 0.005 mg/mL (5 μg/mL) at a pH of 6.5 and 25° C.

The active agent should be understood to include the unionized form of the active agent, pharmaceutically acceptable salts of the active agent, or any other pharmaceutically acceptable forms of the active agent. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms, co-crystals, and prodrugs.

Examples of classes of active agents include, but are not limited to, compounds for use in the following therapeutic areas: antihypertensives, antianxiety agents, antiarrythmia agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol-reducing agents, cholesteryl ester transfer protein inhibitors, triglyceride-reducing agents, antiobesity agents, autoimmune disorder agents, anti-impotence agents, antibacterial, anthelmintics, antihelminthics, antifungal agents, hypnotic agents, anti-Parkinsonism agents, anti-Alzheimer's disease agents, antibiotics, anti-angiogenesis agents, anti-glaucoma agents, anti-depressants, bronchodilators, glucocorticoids, steroids, and mixtures thereof.

In various embodiments, active agents for use in the process and products of the disclosure depend on the target state of matter. In one embodiment, the small active-rich domains are substantially in the amorphous state. In this embodiment the amorphous form of the active may be relatively stable. In another embodiment, the actives are those for which no crystalline state has been observed. In this embodiment, crystalline states may be known but some ionized states and ion pairs may not be known to crystallize. In other cases the amorphous form may be stable because the free energy of crystallization is very low.

Matrix Materials

The process of the present disclosure includes at least one matrix material. By “matrix material” is meant a material consisting of one or more pharmaceutically acceptable excipients in which the small active domains are dispersed. In general, the matrix material is chosen such that the small active domains formed by the process of the present disclosure have the desired size and physical state. In addition, the matrix material can aid in keeping the small active domains from aggregating and upon dissolution in a dosing vehicle or a use environment such as gastro-intestinal (GI) fluid, lung fluid, or plasma, the matrix material can aid in the dissolution process. The matrix material typically constitutes from 0.1 wt % to 99 wt % of the combined mass of the active agent and matrix material by weight of dried particle. When it is desirable for the matrix material to prevent aggregation of the active domains into larger aggregates, the matrix material constitutes more than 20% or even more than 40% of the combined mass of the active agent and matrix material.

The matrix material may be selected from materials having low molecular weight particularly for inhalation applications, however, it is not necessary for it to be so. Exemplary matrix materials include polyvinyl pyrrolidone (PVP), polyethyleneoxide (PEO), polyethylene glycol (PEG), poly(vinyl pyrrolidone-co-vinyl acetate) (PVP-VA), polyoxyethylene-polyoxypropylene block copolymers (also referred to as poloxamers), polymethacrylates, polyoxyethylene alkyl ethers, polyoxyethylene castor oils, polycaprolactam, polylactic acid, polyglycolic acid, poly(lactic-glycolic)acid, lipids, cellulose, pullulan, dextran, maltodextrin, hyaluronic acid, polysialic acid, chondroitin sulfate, heparin, fucoidan, pentosan polysulfate, spirulan, hydroxypropyl methyl cellulose acetate succinate (HPMCAS), hydroxypropyl methyl cellulose propionate succinate, hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, carboxymethyl ethylcellulose (CMEC), hydroxypropyl methylcellulose (HPMC), hydroxypropyl methylcellulose acetate phthalate (HPMCAP), hydroxypropyl methylcellulose propionate phthalate, hydroxypropyl methylcellulose acetate trimellitate (HPMCAT), hydroxypropyl methylcellulose propionate trimellitate, cellulose acetate succinate (CAS), methyl cellulose acetate succinate (MCAS), dextran, dextran acetate, dextran propionate, dextran succinate, dextran acetate propionate, dextran acetate succinate, dextran propionate succinate, dextran acetate propionate succinate, poly(methacrylic acid-co-methyl methacrylate) 1:1 (e.g., Eudragit® L100, Evonik Industries AG), poly(methacrylic acid-co-methyl methacrylate) 1:2 (e.g., Eudragit® S100), poly(methacrylic acid-co-ethyl acrylate) 1:1 (e.g., Eudragit® L100-55), and mixtures thereof. The foregoing list is not intended to indicate that all embodiments are equivalent and/or equally suitable.

In one embodiment, the matrix material comprises components with a molecular weight of less than 10,000 Daltons, less than 5000 Daltons, or even less than 2000 Daltons.

Examples of classes of matrix materials include sugars, sugar alcohols, polyols (as exemplified above), polyethers (as exemplified above), cellulosic polymers (as exemplified above), amino acids, salts of amino acids, peptides, organic acids, salts of organic acids, and mixtures thereof. Specific examples of sugars and sugar alcohols include, but are not limited to fructose, glucose, lactose, mannitol, trehalose, sucrose, raffinose, maltitol, lactitol, sorbitol, xylitol, erythritol, xylose, acorbose, melezitose, galactose, melibrose, isomaltose. Natural sugar extracts, including, but not limited to malt beet sugar, corn sugar, high-fructose corn syrup, and sugar oligomers, such as polydextrose and dextrans with molecular weights less than 10,000 Daltons. Polyols such as glycerol, sorbitol, ethylene glycol, propylene glycol, butanediol, and other oligomers. Amino acids and salts of amino acids, such as glycine, leucine, serine, alanine, isoleucine, tri-leucine. Organic acids and salts of organic acids, such as oleic acid, citric acid, tartaric acid, edetic acid, malic acid, sodium citrate, and mixtures thereof. Low molecular-weight oligomers are suitable including polyethylene glycols, poly amino acids or peptides and copolymers such as polyethylene glycol/polypropylene glycol copolymers, poloxamers, and mixtures thereof. In one embodiment, the matrix material is selected from fructose, glucose, lactose, mannitol, trehalose, sucrose, raffinose, maltitol, lactitol, sorbitol, xylitol, erythritol, xylose, acorbose, melezitose, galactose, melibrose, isomaltose, malt beet sugar, corn sugar, high-fructose corn syrup, polydextrose, and dextrans with molecular weights less than 10,000 Daltons, glycerol, ethylene glycol, propylene glycol, butanediol, glycine, leucine, serine, alanine, isoleucine, tri-leucine, oleic acid, citric acid, tartaric acid, edetic acid, malic acid, sodium citrate, low molecular-weight polyethylene glycols, poly amino acids, polyethylene glycol/polypropylene glycol copolymers, poloxamers, and mixtures thereof. In another embodiment, matrix material is selected from fructose, glucose, lactose, mannitol, trehalose, sucrose, raffinose, maltitol, lactitol, sorbitol, xylitol, erythritol, xylose, acorbose, melezitose, galactose, melibrose, isomaltose, malt beet sugar, corn sugar, high-fructose corn syrup, polydextrose, and dextrans with molecular weights less than 10,000 Daltons. In still another embodiment, the matrix material is selected from glycine, leucine, serine, alanine, isoleucine, tri-leucine, oleic acid, citric acid, tartaric acid, edetic acid, malic acid, sodium citrate, and mixtures thereof. When a crystalline form, the matrix material is preferably selected from the group consisting of lactose, mannitol, trehalose, and mixtures thereof.

Characterization of the Compositions

The compositions of the disclosure may comprise an active-rich domain and an active-poor domain, preferably distinct active-rich and active-poor domains. In one embodiment, the composition comprises solid particles, each of the particles comprising a multiplicity of drug/active-rich domains dispersed in an active-poor domain. In general, these active-poor domains consist primarily of a matrix material.

In one embodiment, the size of the active-rich domains has an average diameter of less than 5 microns. In another embodiment, the average diameter of the active-rich domains can be less than 2 microns, less than 1 micron, or even less than 0.7 microns.

In one embodiment, the active-rich domain is crystalline. Crystalline materials can be identified using modulated Differential Scanning Calorimetry (mDSC), Powder X-Ray Diffraction (PXRD), transmission electron microscopy (TEM), scanning electron microscopy (SEM).

In one embodiment the active-rich domains are amorphous. The amorphous character of the active-rich domains can be determined by the PXRD pattern. Amorphous active-rich domains will generally show only broad diffraction peaks often described as the amorphous halo. In some cases the mDSC scan of the product will show a glass transition associated with the amorphous form of the drug. As the active content of the amorphous active-rich domains approach pure active, the Tg of these domains will approach the Tg of pure amorphous active, meaning within 30° C. of the pure amorphous active.

In other embodiments the active-rich domains are crystalline. By crystalline is meant that either 1) the PXRD of the composition displays scattering peaks that are sharper and narrower than those displayed by amorphous active, or 2) the mDSC scan of the composition displays an endothermic heat flow that is associated with the active rich domains.

In one embodiment, the active in the composition exhibits a powder x-ray diffraction (PXRD) pattern that is different from a PXRD pattern of the active agent in crystalline form. In another embodiment, the PXRD pattern of the composition has at least one peak that has a full width at half height of at least 1.1-fold that of an equivalent peak exhibited by the drug in a control composition. In still another embodiment, the composition has a glass transition temperature that is different than the glass transition temperature of the active agent. In still another embodiment, the composition exhibits an onset or maximum in the melt endotherm that is at a lower temperature than the onset or maximum in the melt endotherm of said active agent in crystalline form.

Methods of Administration

In one embodiment, the disclosure provides a method of treating an animal, including humans, in need of therapy comprising administering a composition comprising an active agent and a matrix material to an animal via a mode selected from the group consisting of oral, buccal, mucosal, sublingual, intravenous, intra-arterial, intramuscular, subcutaneous, intraperitoneal, intraarticular, infusion, intrathecal, intraurethral, topical, subdermal, transdermal, intranasal, inhalation, pulmonary tract, intratracheal, intraocular, ocular, intraaural, vaginal, and rectal.

In one embodiment, the composition comprising an active agent and a matrix material is intended for oral, buccal, mucosal, or sublingual delivery. In this embodiment, the composition may be in the form of a powder that is incorporated into a suitable oral dosage form, such as tablets, capsules, caplets, multiparticulates, films, rods, suspensions, powders for suspension, and the like. Alternatively, the composition may be granulated prior to incorporation into a suitable dosage form.

In another embodiment, the composition comprising an active agent and a matrix material is intended for intravenous, intra-arterial, intramuscular, subcutaneous, intraperitoneal, intraarticular, infusion, intrathecal, intraocular, or intraurethral delivery. In this embodiment, the composition may be in the form of a suspension or solution, suitable for injection via a needle, for introduction to an IV bag or bottle, or delivered via an appropriate catheter to the intended delivery site. In one embodiment, the composition is formulated as a dry powder or solid, that is then reconstituted into a suspension or solution prior delivery. Formulating the composition as a dry powder or solid typically improves the chemical and/or physical stability of the composition. The dry powder or solid is then mixed with a liquid, such as water suitable for injection or other liquid, to form a suspension or solution that may then be delivered via the chosen route. In still another embodiment, the composition is delivered in the form of a depot that controls or otherwise modifies the rate of release of active agent from the depot. The depot may be formed prior to delivery, or may be formed in situ after delivery. Such depots can be in the form of suspensions or can be in the form of a monolith such as a film or rod. The active agent may be released very rapidly by dissolution of the composition when a soluble or enteric or dispersible form of the matrix is used. Alternatively, the active agent may be released over hours, days, or even many months by utilizing a poorly aqueous soluble matrix.

In another embodiment, the composition comprising an active agent and a matrix material is intended for topical delivery. In this embodiment, the composition may be formulated into appropriate creams, transdermal patches, and the like, as is well-known in the art.

In another embodiment, the composition comprising an active agent and a matrix material is intended for inhalation. As used herein, the term “inhalation” refers to delivery to a patient through the mouth and/or nose. In one embodiment, the dry powder suitable for inhalation is delivered to the “upper airways.” The term “upper airways” refers to delivery to nasal, oral, pharyngeal, and/or laryngeal passages, including the nose, mouth, nasopharynx, oropharynx, and/or larynx. In another embodiment, the dry powder suitable for inhalation is delivered to the “lower airways.” The term “lower airways” refers to delivery to the trachea, bronchi, bronchioles, alveolar ducts, alveolar sacs, and/or alveoli.

In one embodiment, the particles have a mass median aerodynamic diameter (MMAD) of 5 to 100 μm. In another embodiment, the particles have a MMAD of 10 to 70 μm. In yet another embodiment, the particles have an average diameter of 50 μm. In one embodiment, such particles are used in devices designed for delivery of particles to the upper airways. In another embodiment, such particles are used in devices designed for delivery of particles via the nose.

In one embodiment, the compositions may be formulated as a dry powder for use in a suitable inhalation device, such as a conventional dry powder inhaler. In another embodiment, the powders may be packaged in a packet suitable for insertion into a dry powder inhaler. Suitable dry powder inhalers typically rely on a burst of inspired air that is drawn through the unit to deliver the powder to the desired location. In another embodiment, the compositions may be administered as aqueous solutions or suspensions, or as solutions or suspensions in propellants, using, for example, a metered-dose inhaler. In this embodiment, the solution or suspension is aerosolized by liquid nebulizers employing either hydraulic or ultrasonic atomization. Compressor-driven nebulizers may also be employed, which may use a suitable propellant.

In another embodiment, the composition comprising an active agent and a matrix material is intended for ocular or intraaural delivery. In this embodiment, the compositions may be formulated into appropriate suspensions, creams, fluids, drops or other suitable forms for administration.

The terms and expressions which have been employed in the foregoing specification are used therein as terms of description and not of limitation, and there is no intention in the use of such terms and expressions of excluding equivalents of the features shown and described or portions thereof, it being recognized that the scope of the disclosure is defined and limited only by the claims which follow.

1. A method for making particulate material comprising the steps of:
providing a first solution comprising one or more solvents and active agents;
providing a second solution comprising an antisolvent, wherein the first solution and/or the second solution further comprises a matrix material;
mixing the first solution with the second solution, to form a mixture;
atomizing the mixture with a gas to produce droplets;
drying the droplets by directing the droplets into a chamber for removal of the solvent and the antisolvent, to produce solid particles;
wherein the atomizing step occurs substantially immediately after the mixing step and is followed by the drying step in a continuous mode, and wherein the mixing step starts at a dedicated position of a spray-drying nozzle 3.

2. The method of claim 1, wherein the atomizing step occurs less than or equal to 500 ms after the mixing step.

3. The method of claim 1, wherein the mixing and the atomizing steps occur at two consecutive positions of the spray-drying nozzle.

4. The method of claim 1, wherein the particles comprise distinct active-rich and active poor domains.

5. The method of claim 1, wherein the solvent consists of an organic solvent.

6. The method of claim 1, wherein the matrix material is crystalline, amorphous, or semi-crystalline.

7. The method of claim 6, wherein the matrix material has a molecular weight of less than 5000 Daltons.

8. The method of claim 1, wherein the matrix material comprises a material selected from sugars, sugar alcohols, polyols, polyethers, cellulosic polymers, amino acids, salts of amino acids, peptides, organic acids, salts of organic acids, and mixtures thereof.

9. The method of claim 1, wherein the antisolvent comprises, preferably consists of, water.

10. A spray-drying nozzle, for use in the production of particulate material, comprising:
a first conduit, having an inlet and an outlet, for conducting a first solution;
a second conduit, having an inlet and an outlet, for conducting a second solution;
a mixing chamber being arranged to permit mixing of the first and second solution to generate a mixture, wherein the mixing chamber is in fluid communication with both said first and second conduits and is located proximal to the outlets thereof;
one or more gas conduit, each having an inlet and an outlet, in fluid communication with an atomization region of the nozzle,
wherein the atomization region is in fluid communication and downstream said mixing chamber and proximal to an outlet orifice of the nozzle, and wherein the one or more gas conduit is arranged to provide a gas flow into the atomization region to atomize the mixture into droplets that are jetted out of the spray-drying nozzle from the outlet orifice in the form of a spray.

11. The spray-drying nozzle of claim 10, wherein the second conduit is positioned coaxially around the first conduit.

12. (canceled)

13. The spray-drying nozzle of claim 11, wherein the one or more gas conduit is positioned coaxially around the second conduit, and preferably merges into the atomization region of the nozzle to permit the gas to atomize the mixture formed in the mixing chamber that flows to the atomization region.

14. The spray-drying nozzle of claim 13, wherein the one or more gas conduit is located on the outermost portion of the nozzle proximal to the outer perimeter thereof.

15. The spray-drying nozzle of claim 14, wherein at least a portion of the first conduit, second conduit and one or more gas conduit, extend substantially parallel to each other along the same axis, and wherein the axis corresponds to the centerline of the nozzle, and wherein the at least a portion is at least a segment proximal to the outlets thereof.

16. The method of claim 1, wherein the atomizing step occurs less than or equal to 150 ms after the mixing step.

17. The method of claim 1, wherein the atomizing step occurs less than or equal to 90 ms after the mixing step.

18. The method of claim 4, wherein the active-rich domains are crystalline, amorphous, or semi-crystalline and have an average diameter of less than 5 microns.

19. The method of claim 8, wherein the matrix material consists essentially of, a material selected from sugars, sugar alcohols, polyols, polyethers, cellulosic polymers, amino acids, salts of amino acids, peptides, organic acids, salts of organic acids, and mixtures thereof.

20. The method of 8, wherein the matrix material is selected from the group consisting of lactose, mannitol, trehalose, and mixtures thereof.

21. The spray-drying nozzle of claim 11, wherein the second conduit is tapered such that the diameter of the second conduit at a position proximal to the outlet of the second conduit and the mixing chamber is less than the diameter at a position distal from the mixing chamber and proximal to the inlet of the second conduit, and wherein both first and second conduits merge into the mixing chamber to permit mixing of the first and second solutions in the mixing chamber.

22. The spray-drying nozzle of claim 15, wherein the second conduit and the one or more gas conduit are tapered such that the diameter at the outlet thereof is less than the diameter at the inlet thereof.
